(19) United States
(12) Patent Application Publication
(10) Pub. No.: US 2022/0244272 A1
(43) Pub. Date: Aug. 4, 2022

(54) In vitro test method for early detection of endometriosis and/or uterine adenomyosis

(71) Applicant: Stefan KISSLER, Frankfurt am Main (DE)

(72) Inventor: Stefan KISSLER, Frankfurt am Main (DE)

(21) Appl. No.: 17/612,880

(22) PCT Filed: May 19, 2020
(86) PCT No.: PCT/EP2020/063924

§ 371 Date: Nov. 19, 2021

(30) Foreign Application Priority Data
May 23, 2019 (DE) 10 2019 113 723.3

(51) Int. Cl. G01N 33/68; G01N 33/543

(52) U.S. Cl. CPC G01N 33/6893; G01N 2800/364; G01N 2333/723; G01N 33/5436

(57) ABSTRACT

An in vitro test method for early detection of endometriosis and/or uterine adenomyosis in a female patient, comprising the following steps:
a) providing menstrual blood of the patient to be tested,
b) determining expression of the genes ESR2 and/or CXCL12 and/or CXCR4 in comparison with at least one control sample,
wherein an increased expression of one or more of the genes indicates endometriosis and/or uterine adenomyosis.

The present invention relates to an in vitro test method for early detection of endometriosis and/or uterine adenomyosis in a female patient.

Endometriosis is one of the most common gynaecological diseases, with a prevalence of 10-15% in all women of reproductive age. It is defined as the occurrence of endometrial stroma—and gland cells—outside the corpus uteri (body of the uterus). Its main symptoms are not only acyclic vaginal bleeding depending on the location, but also pain during urination and defecation and pain during sexual intercourse. Its cardinal symptom is the severe pain during menstruation (dysmenorrhoea), which very often entails the intake of high doses of analgesic.

Adenomyosis is a special form of endometriosis, in which endometrial islands appear within the myometrium (uterine musculature).

The consequences of endometriosis, which is frequently only diagnosed after many years, are extremely unpleasant for the patient: for instance, it is possible that an instance of endometriosis that is painful right from the start undergoes continuous further development and pelvic structures become severely affected thereby. Such disease development is also referred to as “deep-infiltrating endometriosis” or “DIE”. This is frequently then followed by a history of surgical measures on the young woman. Besides the obvious stresses for the patient, said measures are also associated with economic strains on the health system and in some cases with the patient being unable to work for a long time. A further consequence that is highly undesirable is the far-reaching infertility of the woman affected, which is normally associated with many years of endometriosis. In one population of infertility patients, the most common somatic diagnosis made by Kissler et al., with an incidence of 35-40%, was endometriosis or adenomyosis that had been undetected prior to examination (Kissler S, Marx K, Scholtes M, Pfeiffer S, Meier W, Neulen J. Predisposition of subtle endometriotic lesions predominantly on the left side assessed by transvaginal hydrolaparoscopy (THL) Eur J Obstet Gynecol Reprod Biol 2011 October; 158 (2): 185-88).

Dysmenorrhoea, as the cardinal symptom of endometriosis, is characterized by an increased amplitude and frequency of internal pressures on the non-pregnant uterus during menstruation (Buletti C, de Ziegler D, Polli V, del Ferro E, Palini S, Flamigni C. Characteristics of uterine contractility during menses in women with mild to moderate endometriosis. Fertil Steril. 2002 June; 77(6): 1156-61.).

The causes giving rise to endometriosis have still not been completely clarified. Studies which included the involvement of the inventor of the present application clearly point to a link between wound-healing processes as a result of auto-traumatization and the emergence of endometriosis. The wound-healing processes observed and the associated molecular events are of a non-organ-specific nature.

For years, the gold standard in the diagnosis of endometriosis has been laparoscopy (LSC), with an accurate visual inspection of the typical sites of predilection in the lesser pelvis of the woman for the location of pelvic endometriosis. In the event of visual detection, histological detection is carried out via a biopsy (removal of sample tissue). Laparoscopy is an invasive procedure that can represent a stressful examination. Experience shows that laparoscopy carried out on a woman of a very young age frequently gives no signs of pelvic endometriosis or only very weak signs thereof. Accordingly, it has additionally been shown that, for these reasons, a latency period of on average 7-10 years passes by until the treating gynaecologist makes a clear diagnosis by laparoscopy. In many cases, the patient is then already exhibiting infertility, which is only detected in connection with an unfulfilled desire to have children.

In many cases, the cardinal symptom of endometriosis, dysmenorrhoea, is not given the necessary attention by the gynaecologist right away, since the dysmenorrhoea usually starts very early (primary), i.e. with the onset of menstruation (menarche), or only a short period of time later (secondary) after menarche. For such young women or girls, the attending gynaecologist usually has no wish to immediately recommend a laparoscopy, since it is an invasive procedure and is thus also often refused. If, as part of a laparoscopy, pelvic endometriosis or uterine adenomyosis is then established after a disease process of many years, what has generally already started is destruction of the delicate inner muscle layers. Said muscle layers are, however, necessary for uterine contractility and directed transport of sperm. A functional impairment means a decline in fertility can be expected.

Recently, it has been shown that the duration of existing dysmenorrhoea as the cardinal symptom of endometriosis has a positive correlation with the manifestation of uterine adenomyosis (Kissler et al. 2, Parker et al. 3). In the case of long-standing endometriosis, about 80% of women exhibit wall-related changes to the uterine architecture. With respect to diagnostic instruments, transvaginal ultrasound examination or T2-weighted magnetic resonance imaging can be used to achieve this detection. What can be found here are diffuse or local instances of thickening of the uterine wall, changes in the wall texture or thickening of the subendometrial layers (HALO, junctional zone). These uterine changes are not reachable for the surgeon using invasive surgery for diagnosis of endometriosis and therefore remain undetected without additional examinations using imaging methods.

WO2016011377 describes an in vitro test for detection of endometriosis, in which the quantity of transcripts of one of the genes CCL3LI, CCL3, FAM180A, THBS2, PDGFRL, FNI, CLEI IA, CCNA2, KIF20A, BUBIB, HSDI 7B6, HSDI IBI, C7, C3, CXCL2, CXCL12, CXCL13, PDGFC, CXCL14, ACTA2, TAGLN, or SORBSI in tissue samples is measured. The method proposed in WO2016011377 requires tissue removal, for example a biopsy.

DE10048633 describes an in vitro test for detection of endometriosis, in which the quantity of transcript or protein gene product of at least one of the genes fibronectin, insulin-like growth factor binding protein 2, transmembrane receptor PTK7, platelet-derived growth factor receptor alpha, collagen type XVIII alpha 1, subtilisin-like protein (PACE4), laminin M chain (merosin), elastin, collagen type IV alpha 2, p27 interferon alpha-inducible gene, reticulocalbin, aldehyde dehydrogenase 6, gravin, nidogen and phospholipase C epsilon in a patient sample is determined and is compared with a control sample. According to DE10048633, a lower quantity of the gene product indicates the existence of endometriosis. The samples analysed come from an endometrial biopsy (curettage).

JP2009168646 proposes a biomarker for detection of endometriosis in blood serum, which biomarker has a molecular weight of 5830 and is detected using SELDI-TOF mass spectrometry. According to JP2009168646, the sensitivity and specificity of detection are each at least 80%.

It is an object of the present invention to further facilitate the diagnosis of endometriosis and its special form adenomyosis. More particularly, it is an object of the present invention to develop a screening test for women, especially for young women, in order to be able to detect endometriosis and its special form adenomyosis as early as possible. It is desirable here to provide a test method which has high specificity and which can be carried out in a simple manner and without stressful interventions into the woman's body. It is intended that the test method also be suitable for mass screenings. It would therefore then be possible, through suitable treatment concepts, to avoid the further spread of endometriosis as far as its full picture with very severe pains and an unfulfilled desire to have children.

The object is achieved by the proposal of an in vitro test method according to claim 1 for early detection of endometriosis and/or uterine adenomyosis in a female patient.

According to the invention, a detection kit is further proposed, as are PCR primers and antibodies suitable for the purposes of detection.

The in vitro test method proposed by the inventor of the present application for early detection of endometriosis and/or uterine adenomyosis in a female patient comprises at least the following steps:

a) providing menstrual blood of the patient to be tested,

b) determining expression of the genes ESR2 and/or CXCL12 and/or CXCR4 in comparison with at least one control sample, wherein an increased expression of one or more of the genes indicates endometriosis and/or uterine adenomyosis.

ESR2 is the name of the gene encoding “oestrogen receptor beta” (ER-beta or ERβ, ESR2 gene, gene ID 2100).

CXCL12 is the name of the gene encoding “stromal cell-derived factor 1”, a cytokine (gene ID 6387).

CXCR4 is the name of the gene encoding “SDF-1 receptor” (gene ID 7852).

From studies of the expression of the genes ESR2, CXCL12 and CXCR4 in samples of menstrual blood, there were indications that an increased expression of said genes may point to early stages of endometriosis, especially in patients suffering from dysmenorrhoea.

This diagnostic method can be carried out without stressful removal of tissue. The method is comparatively cost-effective and can be used for mass screenings.

The menstrual blood provided in step a) can, for example, be obtained by means of a vaginal examination, especially by means of a gynaecological speculum examination of the patient. Alternatively, the menstrual blood can come from a tampon which has been used by the patient and which, immediately after removal, is frozen at at least −25° C. and/or is placed into a special container in which a suitable preservation liquid is present. Alternatively, what can also be used for the diagnostic method is menstrual blood which has been collected by means of a vaginal cap and has been stabilized by means of a preservation liquid or frozen at at least −25° C.

The menstrual blood can come from a vaginal examination of the patient that was carried out on any of cycle days 1 to 3, preferably on cycle day 2. If the in vitro test is carried out using a tampon provided by the patient, the tampon should also have been used on any of cycle days 1 to 3, preferably on cycle day 2. The same applies to use of a vaginal cap.

The in vitro test method can be used particularly advantageously for patients suffering from dysmenorrhoea, especially for those patients being gynaecologically treated in this respect, since a particularly high specificity of the test can be expected for this patient group.

There is no guideline-based score for classification of dysmenorrhoea. However, in the gynaecological practice of the inventor of the present application, the classification reproduced below has been developed and introduced for quantification of dysmenorrhoea on the basis of the subjectively perceived severity of the patients' sensation of pain:

Dysmenorrhoea 0°: there is no pain during menstruation.

Dysmenorrhoea I°: mild to moderate pain during menstruation with no intake of painkillers.

Dysmenorrhoea II°: moderate to severe pain during menstruation with no intake of painkillers.

Dysmenorrhoea III°: severe pain during menstruation with intake of painkillers.

The in vitro test method should preferably be used for patients who can be assigned to group II° or III°, especially to group III°, according to the classification described here. For patients of group III°, the prospects of a successful treatment of endometriosis or adenomyosis can be rated as good if the treatment is commenced in time, and so said patients particularly benefit from the early detection test.

Regarding the diagnosis of dysmenorrhoea, reference is made at the same time to the ICD (International Statistical Classification of Diseases and Related Health Problems), reference being made here to ICD-10. ICD N 94-4 characterizes primary dysmenorrhoea, N 94-5 secondary dysmenorrhoea.

Accordingly, the invention proposed here according to a particularly preferred embodiment is directed to an in vitro test method for early detection of endometriosis and/or uterine adenomyosis in a female patient, comprising the steps of a) providing menstrual blood of the patient to be tested, and b) determining expression of the genes ESR2 and/or CXCL12 and/or CXCR4 in comparison with at least one control sample, wherein an increased expression of one or more of the genes indicates endometriosis and/or uterine adenomyosis, and wherein the patient is acutely suffering from dysmenorrhoea and/or has a previous history of dysmenorrhoea, especially primary dysmenorrhoea (ICD 94-4) or secondary dysmenorrhoea (ICD 94-5), and/or who can be assigned to class II° or III°, especially to class III°, according to the classification system described in the present application.

According to the findings of the inventor of the present application, dysmenorrhoea might be a trigger for the emergence of endometriosis. The increased contractility of all layers of the uterine wall leads to the auto-traumatization of the uterine musculature mentioned at the start, especially in the region of the evolutionarily oldest functional unit of the uterus, the endometrial-myometrial junction or the region of the so-called basal endometrium. This process, which starts early in the young women affected, proceeds continuously every month, with desquamation and expulsion of fragments of basal endometrium during menstruation. This phenomenon has not been observed in symptom-free women with no existence of dysmenorrhoea. The auto-traumatization of the uterine musculature initiates cellular and molecular biological processes of wound healing that occur ubiquitously in the human body, the consequence of said processes being increased steroidogenesis of the endometrial stromal cells, which then leads to increased paracrine production of oestradiol (Leyendecker G, Wildt L, Mall G. The pathophysiology of endometriosis and adenomyosis: tissue injury and repair. Arch Gynecol Obstet. 2009 October; 280(4): 529-38). Oestradiol plays a leading role in increasing uterine contractility and therefore supports the process of auto-traumatization.

Oestradiol binds to oestrogen receptor beta (ER-beta). Previous work by the inventor of the present application indicated a link between increased ER-beta expression in the desquamated menstrual blood and the appearance of dysmenorrhoea (Kissler S, Schmidt M, Keller N, Wiegratz I, Kohl J, Baumann R, Kunz G, Kaufmann M, Leyendecker G Real-Time PCR-Analyse für Östrogen-Rezeptor beta, Pro gesteronrezeptor and P-450-Aromatase im Menstrualblut—eine Pilotstudie über die Bedeutung des basalen Endometriums in der Pathogenese der Endometriose [Real-time PCR analysis of oestrogen receptor beta, progesterone receptor and P-450 aromatase in menstrual blood—a pilot study on the significance of the basal endometrium in the pathogenesis of endometriosis]. Geburtshilfe Frauenheilkd 2007; 67-A24). Further research work showed that ER-beta is a precursor for the chemoattraction of the chemokine CXCL12. Together with the receptor CXCR4 expressed on the surface of mesenchymal stem cells (MSC), ER-beta and CXCL12 form a cohesive “morphogenetic complex”.

In the wound area which emerges as a result of the increased uterine contractility of the woman suffering from dysmenorrhoea, there is therefore an accumulation of mesenchymal stem cells, and so the earliest emergence of endometriosis could be understood as a uterine disorder with activation of the mesenchymal stem cell system.

The altered expression patterns captured in the in vitro early test disclosed herein are possibly the first signs detectable by molecular biology of an incipient process of destruction.

It is recommended that the in vitro test method be preferably used for patients who are of 18 to 35 years of age or who, at the time of test performance, have never been pregnant (“nulligravidae”). Preferably, the patients are 18 to 30 years of age and nulligravidae. It is precisely in this patient group that attempts can advantageously be made by early treatment to suppress the further spread of endometriosis.

Moreover, it is assumed by the inventor of the present application that the specificity of the test method is improved if the patient to be tested has not undergone hormone therapy within 3 months before menstrual blood collection. In addition, no painkillers should be taken at least one week before menstrual blood collection in order to avoid test interference.

For the sample to be tested and for the control sample(s), the expression of the genes ESR2, CXCL12 and CXCR4 should preferably be determined in comparison with a constitutively expressed housekeeping gene, for example c-Abl, so that it is possible to normalize expression.

Particularly preferred embodiments (i), (ii) or (iii) of the invention are directed to determining, in step b) of the in vitro test method for early detection of endometriosis and/or uterine adenomyosis, (i) simultaneously the expression of the genes ESR2 and CXCL12 or (ii) simultaneously the expression of the genes ESR2 and CXCR4 or (iii) simultaneously the expression of the genes CXCL12 and CXCR4. In said embodiments, only an increase in the expression of the two genes ESR2 and CXCL12 (embodiment (i)) or ESR2 and CXCR4 (embodiment (ii)) or CXCL12 and CXCR4 (embodiment (iii)) represents a positive test result, i.e. an indication of the existence of endometriosis and/or uterine adenomyosis. According to a further particularly preferred embodiment (iv), the expression of the genes ESR2, CXCR4 and CXCL12 is simultaneously determined. In connection with said embodiment (iv), only an increase in the expression of all three genes ESR2, CXCR4 and CXCL12 is judged to be a positive test result.

Furthermore, it is proposed that an increase in expression of relevance to making a diagnosis can be assumed when the expression is increased by at least 1.5-fold, preferably 2.0-fold, particularly preferably 2.5-fold, compared to the at least one control sample. If more than one control sample is used in the test, average values of the expression in the control samples can be used for the purposes of comparison.

The control samples should preferably come from the samples of menstrual blood from women who have no history of dysmenorrhoea and for whom there is no laparoscopically detectable endometriosis or uterine adenomyosis. Furthermore, the donors of the control samples should preferably be nulligravidae and be of 18-35 years of age upon sample collection.

In principle, it is possible for the expression of the genes to be analysed to be determined at the mRNA level or at the level of the protein formed. For both methods of determination, a person skilled in the art is aware of a number of highly sensitive and reproducible measurement methods which can all be used for the determination of expression according to method step b).

For protein determination, the in vitro test methods advantageously usable in connection with the invention shall in particular be indicated by Western blotting, an immunoassay, more particularly ELISA, or a protein microarray, though the invention is not restricted to the methods mentioned.

For mRNA determination, what can be applied in particular to the in vitro test methods—without restricting the invention to the methods mentioned in what follows—is Northern blotting, in situ hybridization, an RNAse protection assay, an RNA microarray or preferably PCR, especially quantitative real-time-PCR. Quantitative real-time-PCR, which is very well established and is available in commercial laboratories, is proposed as particularly recommendable. The quantitative real-time-PCR is particularly preferably carried out in a multiplex reaction, with simultaneous amplification of a constitutively expressed housekeeping gene for normalization.

In the quantitative real-time PCR particularly preferred here, mRNA present in the samples is first transcribed into cDNA by means of random hexamer primers in a known manner. The subsequent quantitative real-time PCR can then be carried out by means of Scorpion primers, Lux primers, lanthanide-labelled probes or preferably by means of FRET probes, especially TaqMan probes.

The invention likewise relates to an in vitro test kit for use in the early detection of endometriosis and/or uterine adenomyosis in a female patient on the basis of real-time-PCR. Such a test kit comprises at least one or more oligonucleotide primer pairs homologous to transcribed regions (exons) of the genes ESR2 and/or CXCL12 and/or CXCR4 and also at least one FRET probe homologous to the target transcript(s). Preferably, the test kit comprises further components required for sample preparation and/or for the test reaction, especially an RNA stabilization reagent, a set of reagents for extraction of mRNA from a blood sample, a set of reagents for transcription of mRNA into cDNA and an oligonucleotide primer pair homologous to the transcribed region of a housekeeping gene, especially c-Abl.

Primer pairs can be designed in an exon-linking manner in a manner known per se in order to suppress the amplification of genomic DNA.

The invention likewise encompasses an in vitro test kit for use in the early detection of endometriosis and/or uterine adenomyosis in a female patient on the basis of an immunoassay, especially ELISA. Here, the test kit contains at least one or more antibodies which bind to the gene products of the genes ESR2 and/or CXCL12 and/or CXCR4. The test kit preferably contains further components required for sample preparation and/or for the test reaction, especially a protein stabilization reagent, a set of reagents for extraction of protein from a blood sample and an antibody which binds to the gene product of a housekeeping gene, especially c-Abl, and also suitable detection reagents such as, for example, the secondary antibody.

In the context of the invention, what are proposed furthermore are oligonucleotide primer pairs homologous to the transcripts of the genes ESR2 and/or CXCL12 and/or CXCR4 for use in the early detection of endometriosis and/or uterine adenomyosis in patients. The use mentioned here particularly advantageously relates to early detection of endometriosis and/or uterine adenomyosis in patients who are suffering from dysmenorrhoea and/or have a previous history of dysmenorrhoea, especially if the patients are nulligravidae up until performance of the test and are of 18-35 years of age upon sample collection.

Correspondingly, the invention proposes antibodies which bind to the gene products of ESR2 and/or CXCL12 and/or CXCR4 for use in the early detection of endometriosis and/or uterine adenomyosis in patients, preferably in patients who are suffering from dysmenorrhoea and/or have a previous history of dysmenorrhoea, especially in patients who are nulligravidae up until performance of the test and are of 18-35 years of age upon sample collection.

### FIGURES

FIG. 1 Primers and probe for the target gene CXCL12

FIG. 2 Sequences of the primers and probe for the target gene ESR2

FIG. 3 Bar chart showing the expression of the target genes CXCL12 and ESR2

### EXEMPLARY EMBODIMENT 1—ANALYSIS OF SAMPLES BY MEANS OF QUANTITATIVE REAL-TIME PCR

Samples of menstrual blood were collected from three young nulligravidae (average age: 31 years) suffering from severe analgesic-dependent dysmenorrhoea during a vaginal examination on cycle day 2 (CD 2) in the practice of the principal investigator. All three women were assigned to group III° according to the above-described system for classification of dysmenorrhoea. For two of the three women, there was laparoscopic detection of fresh endometriosis. For the third woman, there were laparoscopic signs of an already long-standing, relatively old endometriosis and possibly adenomyosis.

The controls used were samples of menstrual blood from three young nulligravidae (average age: 31 years) who had no history of dysmenorrhoea and were gynaecologically healthy.

All patients including the control group were not allowed to have taken hormone therapy within 3 months before menstrual blood analysis or to have taken analgesics directly before menstrual blood collection (24 hours).

Menstrual blood containing menstrually desquamated basal endometrium was expelled from the uterus at about 1-2 uterine contractions per minute. In the analysis presented here, the menstrual blood was obtained by means of a gynaecological speculum examination (alternatively, it would also be possible without any problems for the blood to be received via a syringe with sterile irrigation cannula attached or via a groove-containing vaginal speculum). One to two millilitres of the menstrual blood collected was promptly transferred to a sterile commercially available Nunc tube. The samples were stored at −30° C. in the laboratory at the practice immediately after the examination.

The processing of the menstrual blood samples and the expression analyses were carried out as follows:

Total RNA was extracted from the menstrual blood samples by means of the Direct-zol Miniprep Kit (Zymo Research, Irvine, Calif., United States) according to the manufacturer's protocol.

Complementary DNA (cDNA) was then synthesized using the Super-Script™ Reverse Transcriptase (Invitrogen™, Carlsbad, Calif., United States) in combination with random hexamer primers according to details from the manufacturer.

Quantitative PCR (qPCR) was carried out with a 7500 Real Time PCR System (Applied Biosystems™, California, United States) using a TaqMan probe. The target genes were CXCL12 (gene ID: 6387, chromosome 10811.21) and ESR2 (gene ID: 2100, chromosome 14q23.2-q23.3). The quencher used was BBQ, and the reporter fluorescent dye used was 6FAM.

The sequences of the primers and probe for the target gene CXCL12 and the positions of the oligonucleotides on the gene can be seen in FIG. 1. Four primer combinations were used. Here, the mix CXCL-2,1 corresponds to the primer combination CXCL12F/CXCL12As, the mix CXCL-2,2 corresponds to the primer combination CXCL12F/CXCL12Lo, the mix CXCL-2,3 corresponds to the primer combination CXCL12Se/CXCL12As and the mix CXCL12-2,4 corresponds to the primer combination CXCL12Se/CXCL12Lo.

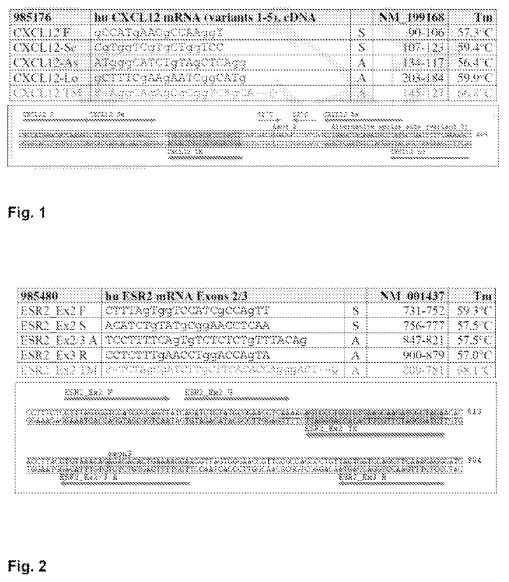

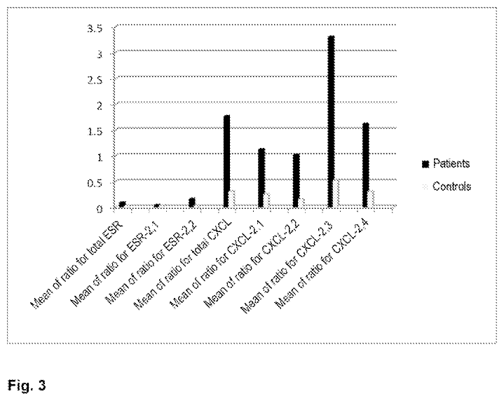

The sequences of the primers and probe for the target gene ESR2 and the positions of the oligonucleotides on the gene can be seen in FIG. 2. In the case of ESR2, two primer combinations were used. Here, the mix ESR-2,1 corresponds to the primer combination ESR2_Ex2 S/ESR2_Ex2/3 A, and the mix ESR-2,2 corresponds to the primer combination ESR2_Ex2 F/ESR2_Ex3 R.

All reactions were carried out in duplicate with a final volume of 30 microlitres using 5× Hot Start Taq Probe qPCR Mix (Axon Labortechnik, Kaiserslautern, Germany).

The following cycles were carried out:

| Cycle No. | Temperature | Duration |
|---|---|---|
| 1st | 50.0° | 2:00 min |
| 2nd | 95.0° | 10:00 min |
| 3rd-45th | 95.0° | 0:05 min |
| | 60.0° | 0:32 min |
| | 72.0° | 0:32 min |

Relative quantification (RQ) of mRNA expression was calculated using the 2(−Delta Delta CT) method (Livak K J, Schmittgen T D. Analysis of relative gene expression data using real-time quantitative PCR and the 2(−Delta Delta C(T)) Method. Methods 2001; 25: 402-408) using a pool of normal samples as calibrator. The reference gene ABL1 (gene ID: 25, chromosome 9q34.12) was used for the normalization of the quantity of mRNA.

### Result:

The relative expression of the genes CXCL 12 and ESR2 for the test group (T) and for the control group (C) is presented in the following table and in FIG. 1.

| | | Control | |
|---|---|---|---|
| | Test group (T) | group (C) | Factor (T)/(C) |
| Mean of ratio for total | 0.092057464 | 0.03465358 | 2.65650677 |
| ESR | | | |
| Mean of ratio for ESR-2,1 | 0.044303902 | 0.03523657 | 1.2573273 |
| Mean of ratio for ESR-2,2 | 0.163687806 | 0.03407058 | 4.80437323 |
| Mean of ratio for total | 1.77088021 | 0.30959724 | 5.71994823 |
| CXCL | | | |
| Mean of ratio for CXCL- | 1.128916467 | 0.25311336 | 4.46012206 |
| 2,1 | | | |
| Mean of ratio for CXCL- | 1.022933426 | 0.15446975 | 6.62222489 |
| 2,2 | | | |
| Mean of ratio for CXCL- | 3.309422411 | 0.52423829 | 6.31282081 |
| 2,3 | | | |
| Mean of ratio for CXCL- | 1.622248534 | 0.30656758 | 5.291650716 |
| 2,4 | | | |

All three patients suffering from severe dysmenorrhoea exhibited distinct overexpression of the CXCL12-ESR2 complex compared to the three control patients. This can be established by the significantly increased rise in the expression pattern in comparison with the housekeeping gene (Ct abl).

The distinct overexpression of essential constituents (CXCL12 and ESR2) of the morphogenetic complex indicates that an invasion and activation of mesenchymal stem cells occurs in the context of uterine wound healing after auto-traumatization. Detection of the overexpression of the gene products of the stated genes from the menstrual blood makes it possible to diagnose the disease endometriosis or adenomyosis at a very early stage.

Accordingly, using the in vitro method presented here, it is possible to develop a screening test for women, especially for young women suffering from severe dysmenorrhoea. In the event of a positive test result, it would be possible to take adequate preventive measures to prevent progression of uterine destruction in endometriosis or adenomyosis. It is also proposed that the screening test be used for quantification of the extent of endometriosis or adenomyosis, even in the case of endometriosis or adenomyosis that has already been diagnosed. Such quantification makes it possible to optimize the options for therapy, especially with a view to treating infertility. Specifically, it is known that both the spontaneous pregnancy rate and the pregnancy rate following assisted reproductive technology (ART) are associated with the severity of uterine adenomyosis in an inversely proportionally manner.

### EXEMPLARY EMBODIMENT 2

The analysis of the expression of the genes ESR2 and CXCL12 that is presented in detail in exemplary embodiment 1 can be carried out in an analogous manner for the gene pair ESR2 and CXCR4 and the gene pair CXCL12 and CXCR4. It is also possible to use the expression of all three genes ESR2, CXCL12 and CXCR4 for the test method.

For the practical implementation of the in vitro test method for early detection of endometriosis and/or uterine adenomyosis in a female patient, it is not only the method described in exemplary embodiment 1 that is of particular significance, but also the analysis of the expression of the genes CXCL12 and CXCR4: patients suffering from severe dysmenorrhoea exhibited distinct overexpression of the CXCL12-CXCR4 complex compared to control patients.

1. In vitro test method for early detection of endometriosis and/or uterine adenomyosis in a female patient, comprising the following steps:
a) providing menstrual blood of the patient to be tested,
b) determining expression of the genes ESR2 and/or CXCL12 and/or CXCR4 in comparison with at least one control sample, wherein an increased expression of one or more of the genes indicates endometriosis and/or uterine adenomyosis.

2. The method according to claim 1, wherein the provided menstrual blood comes from a vaginal examination of the patient that was carried out on any of cycle days 1 to 7 or from a tampon used by the patient.

3. The method according to claim 2, wherein the menstrual blood was frozen at −25° C. or below and/or admixed with a stabilization reagent immediately after the examination or after the removal of the tampon from the body.

4. The method according to claim 1, wherein the patient is acutely suffering from dysmenorrhoea and/or has a previous history of dysmenorrhoea.

5. The method according to claim 4, wherein the dysmenorrhoea can be assigned to ICD 94-4 or ICD 94-5.

6. The method according to claim 4, wherein the dysmenorrhoea can be assigned to class II° or III° according to the classification system described in the present application.

7. The method according to claim 1, wherein the patient is of 18 to 35 years of age and/or is a nulligravida.

8. The method according to claim 1, wherein the patient has not undergone hormone therapy within 3 months before menstrual blood collection.

9. The method according to claim 1, wherein, for the sample to be tested and for the control sample(s), the expression of the genes ESR2 and/or CXCL12 and/or CXCR4 is determined in comparison with a constitutively expressed housekeeping gene, so that it is possible to normalize expression.

10. The method according to claim 1,
wherein a simultaneous increase in the expression of the genes ESR2 and CXCL12, or
a simultaneous increase in the expression of the genes ESR2 and CXCR4, or
a simultaneous increase in the expression of the genes CXCL12 and CXCR4, or
a simultaneous increase in the expression of the genes ESR2, CXCR4 and CXCL12 indicates endometriosis and/or uterine adenomyosis.

11. The method according to claim 1, wherein an increase in expression of relevance to making a diagnosis can be assumed when the expression is increased by at least 1.5-fold, compared to the at least one control sample, wherein, in the case of use of more than one control sample, the average values of the expression in the control samples are used for the purposes of comparison.

12. The method according to claim 1, wherein the at least one control sample comes from the samples of menstrual blood from one or more women who have no history of dysmenorrhoea and for whom there is no laparoscopically detectable endometriosis or uterine adenomyosis, wherein the women are nulligravidae and are of 18-35 years of age upon sample collection.

13. The method according to claim 1, wherein expression is determined on the basis of mRNA determination or on the basis of protein determination.

14. The method according to claim 13, wherein the mRNA determination is carried out by means of Northern blotting, by means of in situ hybridization, by means of an RNAse protection assay, by means of an RNA microarray or by means of PCR.

15. The method according to claim 14, wherein the mRNA is first transcribed into cDNA and subsequently quantitative real-time-PCR is carried out by means of Scorpion primers, Lux primers, lanthanide-labelled probes or FRET probes.

16. The method according to claim 13, wherein the protein determination is carried out by means of Western blotting, by means of a protein microarray or by means of an immunoassay.

17. In vitro test kit for use in the early detection of endometriosis and/or uterine adenomyosis in a female patient on the basis of quantitative real-time PCR, comprising:
one or more oligonucleotide primer pairs homologous to transcribed regions of the genes ESR2 and/or CXCL12 and/or CXCR4;
optionally an RNA stabilization reagent;
optionally a set of reagents for extraction of mRNA from a blood sample;
optionally a set of reagents for transcription of mRNA into cDNA;
optionally at least one oligonucleotide primer pair homologous to the transcribed region of a housekeeping gene, especially c-Abl; and
at least one FRET probe homologous to the target transcript(s).

18. In vitro test kit for use in the early detection of endometriosis and/or uterine adenomyosis in a female patient on the basis of an immunoassay, comprising:
one or more antibodies which bind to the gene products of the genes ESR2 and/or CXCL12 and/or CXCR4;
optionally a protein stabilization reagent;
optionally a set of reagents for extraction of protein from a blood sample; and
optionally at least one antibody which binds to the gene product of a housekeeping gene, especially c-Abl.

19. Oligonucleotide primer pairs homologous to the transcripts of the genes ESR2 and/or CXCL12 and/or CXCR4 for use in the early detection of endometriosis and/or uterine adenomyosis in patients.

20. Antibodies which bind to the gene products of ESR2 and/or CXCL12 and/or CXCR4 for use in the early detection of endometriosis and/or uterine adenomyosis in patients.

21. Primer pairs according to claim 19, wherein the patients have dysmenorrhoea.

22. Primer pairs according to claim 19, wherein the patients are nulligravidae and are of 18-35 years of age upon sample collection.

23. Antibodies according to claim 20, wherein the patients have dysmenorrhoea.

24. Antibodies according to claim 20, wherein the patients are nulligravidae and are of 18-35 years of age upon sample collection.
